(19) United States
(12) Patent Application Publication
(10) Pub. No.: US 2025/0375260 A1
(43) Pub. Date: Dec. 11, 2025

(54) MEDICAL INSTRUMENT WITH POSITIONING X-RAY MARKERS, A COMPUTER-IMPLEMENTED METHOD FOR DETERMINING POSITIONING INFORMATION, AND AN X-RAY DEVICE

(71) Applicant: Siemens Healthineers AG, Forchheim (DE)

(72) Inventors: Alois Regensburger, Poxdorf (DE); Stephan Kellnberger, Zorneding (DE)

(21) Appl. No.: 19/231,548

(22) Filed: Jun. 8, 2025

(30) Foreign Application Priority Data
Jun. 7, 2024 (DE) 10 2024 205 253.1

(51) Int. Cl. A61B 90/00

(52) U.S. Cl. CPC A61B 90/39; A61B 2090/3966; A61B 2560/04

(57) ABSTRACT

A medical instrument for invasive use has an X-ray marker arrangement with at least two X-ray marker areas. The X-ray marker areas are configured to have different absorption properties for X-radiation with different energy spectra, and the X-ray marker areas are arranged successively on the medical instrument with regard to a predetermined spatial direction.

This application claims the benefit of German Patent Application No. DE 10 2024 205 253.1, filed on Jun. 7, 2024, which is hereby incorporated by reference in its entirety.

### BACKGROUND

The present embodiments relate to an instrument for invasive medical use with positioning X-ray markers and a computer-implemented method for determining positioning information. The present embodiments also relate to an X-ray device set up to execute the method and a corresponding computer program product and computer-readable storage medium.

Instruments for invasive medical use may be, for example, guide wires, catheters, endoscopes, laparoscopes, surgical tools, or needles. Needles may be, for example, biopsy needles or ablation needles. Ablation needles (e.g., microwave (MW) or radiofrequency (RF) needles) are used for thermal ablation of tumors among other things. In the case of liver tumors, for example, MW/RF needles are inserted into the human body and into the liver until they reach the tumor. Biopsy needles are used in soft tissue or bone anywhere in the body and are inserted until they reach a tumor or lesion.

Image guidance with CT images is often used when needles are used invasively. CT images make it possible to detect how far the needle is inserted into the body and what the position is of the needle in the body. Image guidance with CT images may be based on the step-and-shoot method. This involves advancing the needle step by step under CT monitoring and checking both the progression and position of the needle between the steps. The CT images available for monitoring are usually thin tomographic images, which are also called slices. The thin tomographic images may include one or more slices.

A practical problem with thin tomographic images arises with the use of so-called “double-oblique” needle trajectories, which are advantageous for the patient at a number of target points. Double-oblique needle trajectories do not usually run within a tomographic image, but rather inclined at a certain angle to the plane of the tomographic image. In such cases, thin tomographic images only show the small longitudinal section of the needle that crosses the spatial area illustrated in the tomographic image. Other longitudinal sections of the needle are outside the tomographic image at both sides. This makes orientation difficult during CT needle guidance, as it is not actually possible to detect which longitudinal section of the needle is illustrated in the tomographic image or how far the needle has already been inserted into the body.

Interventional CT scanners often have laser guidance available, which supports manual guidance of the needle along a planned needle trajectory, which can be helpful with double-oblique trajectories in particular. However, as soon as the needle deviates from the planned trajectory, for example due to deformation of the pierced tissue or due to patient movements, step-and-shoot is once again required.

Another practical problem with thin tomographic images arises with multi-needle procedures (e.g. MW ablations with multiple needles or cryoablations with multiple needles). With multi-needle procedures on larger tumors, precise positioning of all needles is important to ensure complete and precise ablation of the tumor. However, with multi-needle procedures it is often difficult to differentiate the needles from each other in the CT image, which makes orientation difficult and slows down such interventions.

The use of optical or electromagnetic navigation systems for needle guidance is also known. The devices required for this are expensive, however. Additionally, optical navigation in particular can only detect the proximal longitudinal section of the needle located outside the body, but not the distal longitudinal section of the needle. Integrated electromagnetic location elements in the needle tip for detecting the distal longitudinal section of the needle are known. These do have the disadvantage, however, that additional electrical cables and electromagnetic field generators are needed, which can make handling difficult and have an additional space requirement.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, invasive procedures with one or more instruments are supported by determining and displaying positioning information relating to the current position of the respective instrument in each instance, without additional positioning or tracking systems being required.

The present embodiments use spectral X-ray imaging. Spectral X-ray imaging, such as dual-energy CT (DECT), exploits the fact that materials have different absorption properties at different X-ray energies. The absorption properties may also differ from material to material for the respective X-ray energies. With spectral imaging, two different X-ray energy levels are used to produce images. Materials such as bone, soft tissue, or contrast agents absorb X-rays to a different degree, depending on the X-ray energy level. By analyzing X-ray absorption at different X-ray energy levels, specific information may be obtained about the composition of materials or the presence of certain materials. Calcium in vessels may be differentiated from iodine-based contrast agents, for example.

For spectral X-ray imaging, CT scanners with photon-counting detectors are known, for example, or dual-source dual-energy CT scanners, or dual-energy CT scanners with dual-layer detectors. Further, C-arm X-ray devices with two X-ray sources with different X-ray energy levels are also known for spectral X-ray imaging, as well as C-arm X-ray devices with an X-ray source that is toggled between different X-ray energy levels, or toggled between different X-ray filters.

The present embodiments also use an instrument with areas with different spectral X-ray absorption properties (e.g., areas that each have different absorption properties for X-radiation with different energy spectra).

According to the present embodiments, spectral X-ray imaging takes pictures of a layer or a volume, where at least one section of the instrument is within the layer or the volume. The instrument position may then be tracked by spectral position coding along the instrument. Spectral position coding provides that the spectral X-ray absorption properties of the instrument section within the image may be used to determine which section of the instrument is involved. This makes advantageous use of the fact that spectral X-ray images allow for good differentiation of materials with different spectral X-ray absorption properties, which is not possible in a comparable manner with non-spectral X-ray images.

If the spectral X-ray absorption properties of the instrument sections are already known (e.g., from the manufacturer information for the instrument or from a previously taken spectral calibration X-ray image), the knowledge of the section of the instrument also provides that the overall position of the instrument is known. If the spectral X-ray absorption properties of the instrument sections are not already known, it is nevertheless possible to track whether and how often successive sections of the instrument have passed through the image section. If the length of the successive sections is already known, for example, then the distance that the needle has advanced may be concluded from the count of the section change in the image section. If a certain sequence of spectral X-ray absorption properties of the sections of the instrument is already known, the sequence in the image section may be used to conclude when a predetermined needle advance is reached. For example, the spectral X-ray absorption properties may be constant over a certain length of the needle and merely change in a final longitudinal section; then, the change may be used to conclude when the final section has been reached. For example, the spectral X-ray absorption properties may change monotonically over a certain length of the needle and merely change non-monotonically (e.g., inversely) in a final section; then, the inverse change may be used to conclude when the final section has been reached.

A medical instrument for invasive use according to the present embodiments has an X-ray marker arrangement with at least two X-ray marker areas. The X-ray marker areas are configured such that the X-ray marker areas each have different absorption properties for X-radiation with different energy spectra. The X-ray marker areas are arranged successively on the instrument with regard to a predetermined spatial direction.

The present embodiments enable information to be determined relating to the positioning of an invasive instrument in a tomographic image using the different absorption properties of the X-ray marker areas, without the instrument having to be fully shown in the tomographic image and without a separate tracking system being required for this purpose. Providing an instrument according to the present embodiments with spectral X-ray marker areas (e.g., in the form of coatings or a variation in the material composition of the instrument itself) is a simple process. The instrument is also left in its basic design and thus its handling remains advantageously unchanged.

According to one embodiment, the instrument is configured in an elongated form, and the predetermined spatial direction corresponds to the longitudinal direction of the instrument. This enables the respective longitudinal section of the instrument to be detected using the different absorption properties of the X-ray marker areas.

According to an embodiment, the X-ray marker areas each have a different metal content. Metal content may be a varying amount of metal, as well as a varying mixing ratio of metal with other metals or materials in an alloy. In the case of a varying mixing ratio, the amount of metal does not necessarily need to vary. It is only essential that the different metal content results in different spectral absorption properties. Varying the metal content represents a particularly easy and simple option for varying the spectral absorption properties. Additionally, metal is a typical material frequently used in invasive medical instruments.

According to one embodiment, the X-ray marker areas are configured as X-ray markers spatially separated from each other. The spatial separation facilitates particularly good and easy detection of the respective X-ray marker area in X-ray images, as the spatial separation represents a reliably detectable feature in images.

According to one embodiment, the X-ray marker areas are configured as areas of a one-piece X-ray marker. A one-piece embodiment avoids separation areas, which, depending on the embodiment of the X-ray marker areas, may cause surface irregularities at the area boundaries or non-continual changes in the properties and composition of the surface or of the instrument at the area boundaries.

According to one embodiment, the differences in the absorption properties of the X-ray marker areas for X-radiation with different energy spectra change incrementally from X-ray marker area to X-ray marker area. An incremental or gradual change may be detected particularly reliably in spectral X-ray images. The individual stages or acts represent information that may be detected particularly reliably about the respective X-ray marker area in the X-ray image, as the stages may reliably be assigned to a position in the X-ray image.

According to one embodiment, at least one other X-ray marking is provided on the instrument in addition to the X-ray marker arrangement. The at least one other X-ray marking is configured to have different absorption properties for X-radiation with different energy spectra, and the at least one other X-ray marking is arranged such that the at least one other X-ray marking spatially encompasses the X-ray marker arrangement. The other X-ray markings may be configured on an instrument-specific basis. In one embodiment, the X-ray markings may then be used to simply and reliably identify which instrument may be seen in the X-ray image, or to simply and reliably differentiate instruments provided with specific X-ray markings from each other.

### BRIEF DESCRIPTION OF THE DRAWINGS

Other embodiments and advantages arise from the dependent claims and the following description of the exemplary embodiments using the figures.

FIG. 1 shows a needle with sectional spectral X-ray absorption properties;

FIG. 2 shows needles with additional spectral X-ray marking;

FIG. 3 shows an X-ray device;

FIG. 4 is a tomographic image with needle section and positioning information;

FIG. 5 shows a method for executing an embodiment; and

FIG. 6 shows a method for executing an embodiment.

### DETAILED DESCRIPTION

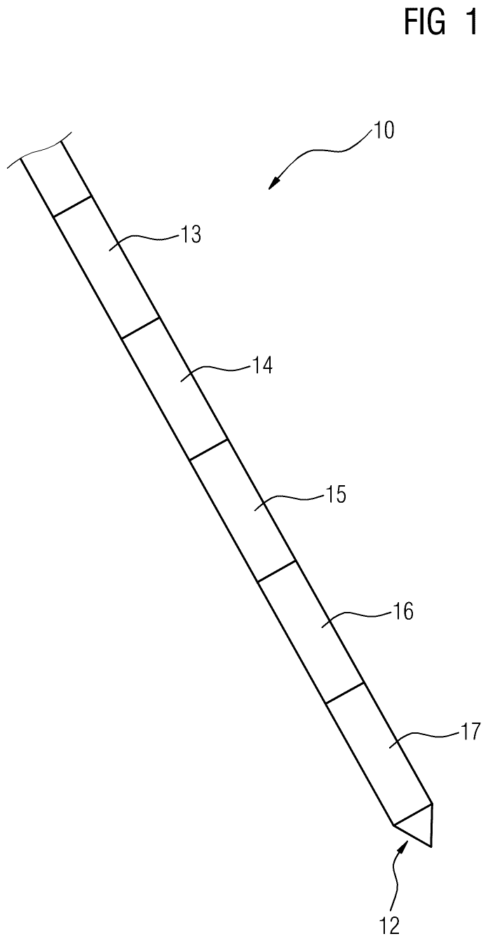

A schematic representation of a needle 10 is shown in FIG. 1 as an example of a medical instrument for invasive use. The needle 10 has a needle tip 12 at a distal end of the needle 10. The needle 10 essentially has a conventional form. The needle 10 may, for example, be a standard MW/RF ablation needle.

According to one embodiment, the needle 10 has a metal or other material mixture or composition varying spatially along a length of the needle 10. The key factor when choosing the material is for the material to have spectral X-ray absorption properties that may be determined with the usual X-ray parameters in medical imaging. In addition to metal, other materials may be considered, such as iodine, calcium, barium, gold, or lead.

The various material compositions may be applied, for example, as a coating on a body of the needle 10. For example, the needle 10 may also be coated with metal or another material of varying thickness. Alternatively, the material used to make the body of the needle 10 may also be varied, for example. Instead of a needle 10, this may also be a bone trocar, for example, that is made from an outer metal tube and an inner carbide pin; the metal tube and/or carbide pin may have a metal or other material composition that varies spatially along a length of the trocar.

Due to differences in the respective metal or material composition or coating, the amounts of the respective metals or materials vary spatially along the length of the needle 10, resulting in spatially varying spectral X-ray absorption properties. The spatially varying spectral X-ray absorption properties form an X-ray marker arrangement of successive X-ray marker areas 13, 14, 15, 16, 17 along the length of the needle 10, which each have different X-ray absorption properties for X-radiation with different energy spectra. The spectral X-ray absorption properties may change incrementally from X-ray marker area 13, 14, 15, 16, 17 to X-ray marker area 13, 14, 15, 16, 17, for example. Alternatively, the spectral X-ray absorption properties may be modulated continually along the length, rather than changing incrementally from area to area.

For example, the “spectral absorption ratio” of the X-ray marker areas 13, 14, 15, 16, 17, defined by dividing the absorption coefficient of the hard X-rays (e.g., X-ray energy >120 keV) by the absorption coefficient of the soft X-rays (e.g., X-ray energy <70 keV) may be, for example, 20% higher in X-ray marker area 13 than in X-ray marker area 14. These spectral X-ray absorption properties may be achieved, for example, through varying metal mixtures of elements such as iron, tantalum, bismuth, copper, and others. Alternatively or additionally, the spectral X-ray absorption properties may also be achieved, for example, using non-metal elements such as carbon, hydrogen, or oxygen as part of the material mixture.

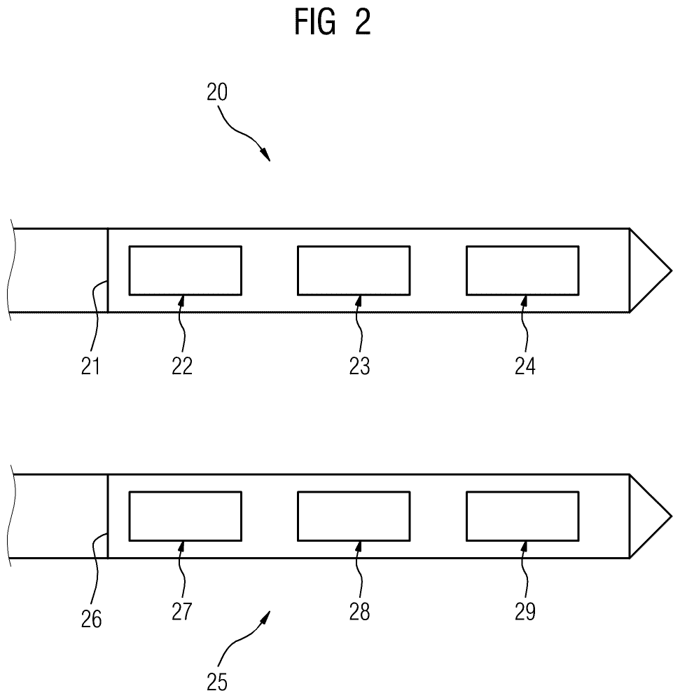

A schematic representation of two needles 20, 25 as invasive instruments is shown in FIG. 2 as an example of another embodiment variant. The needles 20, 25 each have an X-ray marker arrangement of successive X-ray marker areas 22, 23, 24, 27, 28, 29 along the length of the respective needles 20, 25. The X-ray marker areas 22, 23, 24, 27, 28, 29 have different spectral X-ray absorption properties. Reference is made to the preceding figure description in this regard.

Additionally, the needles 20, 25 each have an X-ray marking 21, 26. The X-ray markings 21, 26 are spatially located in the immediate vicinity of the respective X-ray marker areas 22, 23, 24, 27, 28. The purpose of the immediate spatial proximity is that the respective X-ray marking 21, 26 is always included in the image if an X-ray marker area 22, 23, 24, 27, 28 is included in the image in an X-ray tomographic image or an X-ray volume image.

The X-ray markings 21, 26 may, for example, be applied as background coating on the respective longitudinal section of the respective needle 20, 25 and be formed by a layer beneath the respective X-ray marker areas 22, 23, 24, 27, 28. Conversely, the X-ray markings 21, 26 may, for example, also be formed by a layer above the respective X-ray marker areas 22, 23, 24, 27, 28. The X-ray markings 21, 26 may, for example, also be formed by a layer arranged around the respective X-ray marker areas 22, 23, 24, 27, 28.

The X-ray markings 21, 26 have different spectral X-ray absorption properties. This facilitates a differentiation of the X-ray markings 21, 26 and thus of the needles 20, 25. Accordingly, spectral X-rays may be used to determine which of the two needles 20, 25 is shown in the image in each case. This is advantageous for multi-needle procedures. In the case of a procedure with multiple needles 20, 25, needle 20 has an X-ray marking 21 with different spectral X-ray absorption properties to those of the X-ray marking 26 of needle 25. This facilitates a spectral identification of needles 20, 25 and thus their differentiation. The spectral X-ray absorption properties of the X-ray marker areas 22, 23, 24, 27, 28 may also be analyzed at the same time.

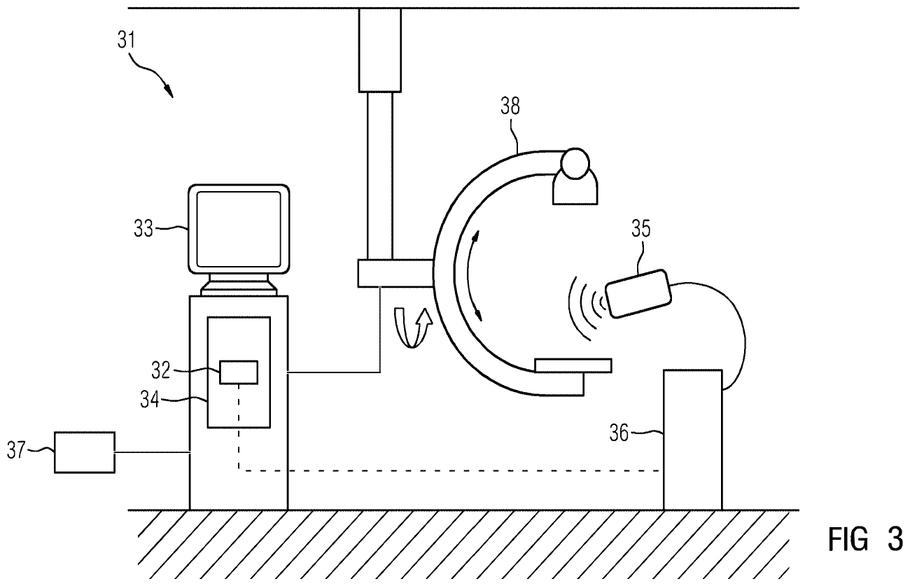

FIG. 3 shows a schematic representation of an X-ray device 31. The X-ray device 31 includes a C-arm 38 with X-ray source and X-ray detector. The C-arm 38 is set up in an essentially known manner to produce spectral X-ray image data sets. For this purpose, the C-arm 38 is able to take X-ray images at different X-ray energy levels. This may be achieved in the known manner (e.g., by changing the X-ray energy of the X-ray source). Alternatively, the arrangement may include two X-ray sources that are operated at different X-ray energy levels. Spectral X-ray image data sets are to be produced.

The X-ray device 31 is also set up in a known manner to produce CT image data sets. For this purpose, the C-arm 38 is able to take multiple 2D projections from different projection angles, by being moved in a circular trajectory around an examination subject (e.g., a patient). CT image data sets (e.g., 3D images) are reconstructed from the 2D projections. The 3D images may be produced as spectral X-ray image data sets in the known manner by the X-ray device 31.

The X-ray device 31 includes a control device 34 that controls the movement of the C-arm 38 and facilitates the operation of the X-ray device 31. The control device 34 includes a computer unit 32 that is set up for image processing and 3D reconstruction. The computer unit 32 is able to produce both 2D X-ray images and 3D image data sets and both non-spectral and spectral images.

The X-ray device 31 also includes a screen 33, on which the images produced by the control device 34 may be displayed.

The control device 34 may optionally receive image data from an ultrasound device 36. The ultrasound device 36 includes an ultrasound head 35 for producing ultrasound images of an examination subject. The ultrasound images may be 2D or 3D images in the known manner. The computer unit 32 is able to combine image data from the ultrasound device 36 with image data from the X-ray device 31 in the known manner (e.g., through fusion or mutual overlapping).

The control device 34 may optionally receive data from a data source 37. The data source 37 may be an integral part of the control device 34 that is integrated in the X-ray device 31. However, the data source 37 may also be provided separately from the control device 34 and merely be connected thereto through a data connection. The data connection may be a wireless or wired connection. The data source 37 may, for example, make available specific information relating to invasive medical instruments. The specific information may, for example, come from the manufacturer of the invasive instruments or be obtained from a calibration X-ray image.

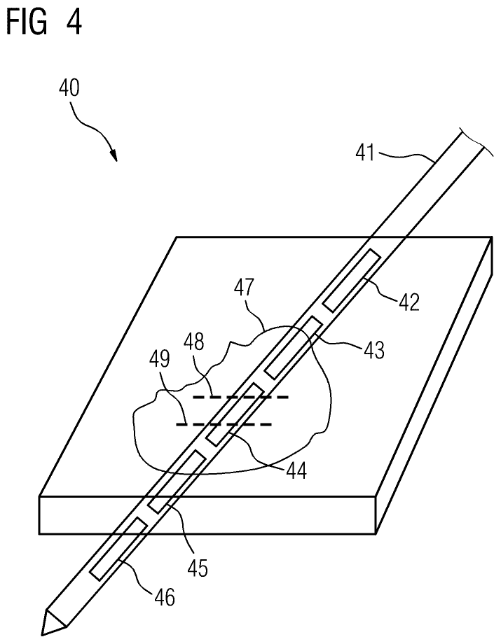

FIG. 4 shows a schematic representation of a tomographic image 40 of a subject body. The tomographic image 40 is a 3D image reconstructed by the X-ray device 34, which represents one layer of an examination subject. Alternatively, the tomographic image 40 may also have been produced by the ultrasound device 36.

The tomographic image 40 includes a schematic illustration of a structure 48. The structure 48 may, for example, be an anatomical organ or a lesion or region with tissue changes.

The figure also includes a schematic representation of an invasive medical instrument (e.g., a needle 41). The needle 41 does not pass within the image plane of the tomographic image 40, but rather at an oblique angle thereto. Thus, it crosses the tomographic image 40. Accordingly, the tomographic image 40 does not show the full length of the needle 41, but merely a short longitudinal section of the needle 41. This is indicated by two dashed lines 48, 49. Only the longitudinal section of the needle 41 between the dashed lines 48, 49 is included in the tomographic image 40. The sections outside the dashed lines 48, 49 are not included in the tomographic image 40 and are merely shown in FIG. 2 for better understanding.

Spectral X-ray absorption properties of the longitudinal section of the needle 41 are determined in the tomographic image 40 according to the present embodiment (e.g., the different X-ray absorption of this longitudinal section is determined for X-radiation at different X-ray energy levels). In order to determine spectral X-ray absorption properties, X-ray images are taken by the X-ray device 34 at different X-ray energy levels. If the tomographic image 40 was produced as a spectral tomographic image 40 from the start, the spectral X-ray absorption properties may be taken directly from the tomographic image 40.

Alternatively, the tomographic image 40 may also be received by the ultrasound device 36, for example. In this instance, the spectral X-ray absorption properties of the longitudinal section of the needle 41 may specifically be determined by the X-ray device 34. The tomographic image 40 may then be registered with the coordinate system of the X-ray device 34. In this manner, the registration may be used for correct spatial assignment of the spectral X-ray absorption properties of the longitudinal section of the needle 41 shown in the tomographic image 40.

The needle 41 has multiple X-ray marker areas 42, 43, 44, 45, 46. The X-ray marker areas 42, 43, 44, 45, 46 exhibit different X-ray absorption properties. The spectral X-ray absorption properties, which are determined by the X-ray device 34 for the longitudinal section of the needle 41 shown in the tomographic image 40, may be used according to the present embodiments to determine positioning information relating to the needle 41. For example, a data source 37 may include an assignment of the specific spectral X-ray absorption properties for the respective X-ray marker area 42, 43, 44, 45, 46 and an assignment of the respective X-ray marker area 42, 43, 44, 45, 46 to its arrangement on the needle 41. For each X-ray marker area 42, 43, 44, 45, 46, the data source 37 may, for example, store the information of how far a respective X-ray marker area 42, 43, 44, 45, 46 is from the tip of the needle 41. In other words, the determined X-ray absorption properties may be used to identify, based on the information obtained from the data source 37, which of the X-ray marker areas 42, 43, 44, 45, 46 is included in the tomographic image 40 and on which longitudinal section of the needle 41 this is arranged.

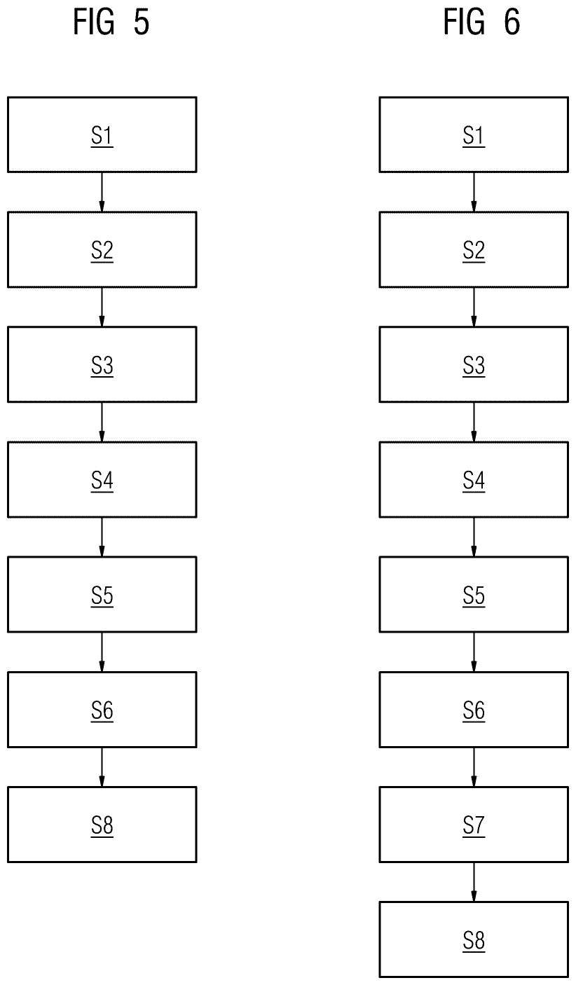

Using the X-ray absorption properties, in FIG. 4, it may be specifically determined that the X-ray marker area 44 is shown in the tomographic image 40. The information on its arrangement on the needle 41 may then be used to specifically determine, for example, how far the X-ray marker area 44 is from the tip of the needle 41. This information may then be used, for example, to specifically determine how far the needle 41 was advanced beyond the area of the examination subject shown in the tomographic image 40, or how far the tip of the needle 41 is from the area of the examination subject shown in the tomographic image 40. A method for executing the present embodiments is explained in FIG. 5.

In act S1, a tomographic image 40 is received by the computer unit 32. The tomographic image may be a spectral CT image. Alternatively, the tomographic image may also be an ultrasound image.

In act S2, an invasive medical instrument or a needle 41 is detected in the tomographic image 40. The instrument or the needle 41 may be detected using a known image processing method (e.g., using a method of pattern recognition).

In act S3, a spatial position of the instrument or the needle 41 is detected in the image.

In act S4, a spectral X-ray image data set that includes at least the previously detected spatial position is produced.

In act S5, a spectral parameter value for the spatial position of the instrument or the needle 41 is determined using the spectral X-ray image data set. In order to be able to use the position from the tomographic image 40, the tomographic image 40 is to be registered with the X-ray device 31 that produces the spectral X-ray image data set. If the tomographic image 40 was produced by the X-ray device 31 itself, it is registered therewith in advance. If the tomographic image 40 comes from another image source, registration is to be effected first. Registration may be effected in the known manner. For example, registration may be effected based on the image data itself, by the tomographic image 40 of the examination subject and of the instrument or the needle 41 being registered by the X-ray device 31 with image data of the examination subject and of the instrument or needle 41 (e.g., using a known image registration method). Known methods register image data, for example, using detectable landmarks in the image data. Landmarks may be, for example, instruments, needles, or anatomical features of the examination subject. Known methods may also register image data by maximizing the similarity between the image data being registered.

In act S6, positioning information assigned to the parameter value is determined. The positioning information includes indirect or direct information relating to the positioning of the instrument or needle 41 shown in the tomographic image 40 relative to the area of the examination subject shown by the tomographic image 40. The determination of positioning information assigned to the parameter value may be based, for example, on the spectral X-ray absorption properties of areas of the instrument or needle 41 and of X-ray marker areas 42, 43, 44, 45, 46 thereof being known in advance. They may be known in advance either as characteristic values of the instrument or needle 41. Or they may be determined and stored in advance based on a previous calibration measurement (e.g., using the X-ray device 31 itself). The spectral X-ray absorption properties may change stepwise or incrementally from area to area. The spectral X-ray absorption properties may also change continually across the areas. The spectral X-ray absorption properties may also change continually within areas and stepwise or incrementally between the areas.

In act S8, the positioning information or information derived therefrom is output via an output device 33. Output via the output device 33 enables an observer to detect how far the instrument or the needle 41 was advanced through the area of the examination subject shown in the tomographic image 40, or how far the tip of the instrument or the needle 41 is from the area shown.

Thus, summarizing the method explained in FIG. 5, if the tomographic image 40 is a layer or a thin volume of a spectral CT image data set, the part of the instrument or the needle 41 intersecting the image is localized through image analysis using a standard instrument or needle detection algorithm, which is known from the prior art. Which X-ray marker area 42, 43, 44, 45, 46 is shown is then determined based on the observed spectral absorption ratio. Further, the angle of inclination of the instrument or the needle 41 relative to the tomographic image 40 may be determined through image analysis. By combining the information about the position of the X-ray marker area or areas 42, 43, 44, 45, 46, which intersect the tomographic image 40, with the detected position and inclination of the instrument or the needle 41, the complete 3D pose and 3D position of the instrument or the needle 41 may be calculated with regard to the tomographic image 40.

Alternatively, if the tomographic image 40 is an ultrasound image, the information about which X-ray marker area 42, 43, 44, 45, 46 of the instrument or the needle 41 is intersecting the ultrasound image plane may be determined based on the spectral absorption ratio. The tomographic image 40 is registered with the coordinate system of the X-ray device 31 for this purpose, or the coordinate system of the ultrasound device 36 is registered with the coordinate system of the X-ray device 31. The registration of the coordinate system may be used to spatially assign the position of the instrument or the needle 41 in the tomographic image 40 to a position in the coordinate system of the X-ray device 31. A spectral X-ray image of the instrument or the needle 41 may then be taken by the X-ray device 31 using the thus assigned spatial position. A locally restricted spectral X-ray image of the position in question is sufficient here, and a complete CT reconstruction is not necessarily required for this. A low number of at least 2 X-ray projections from different projection angles is sufficient for a locally restricted spectral X-ray image. It is also sufficient for a locally restricted spectral X-ray image to have a heavily collimated X-ray beam (e.g., heavily localized to a spatial extent) in order to reduce the X-ray dose.

Regardless of whether the tomographic image 40 is based on an ultrasound image data set or an X-ray or CT image data set, the determined positioning information may also be used advantageously to produce a virtual 3D view of the needle 41 with regard to the tomographic image 40, including the inclination of the needle 41 to the tomographic image 40. Knowledge of the spatial relationship of the needle 41 and tomographic image 40 may also be used when navigating the needle 41 for navigation and image guidance purposes.

A method for executing an embodiment is explained in FIG. 6. The method explained in FIG. 6 corresponds in most method acts to the method explained in FIG. 5. In this respect, the same reference signs are used, and reference is made to the explanations for FIG. 5 to this effect.

Additionally, another parameter value is determined using the spectral absorption properties in act S7. The additional parameter value is assigned to an X-ray marking 21, 26. The X-ray marking 21, 26 is provided on the instruments or needles 41 in addition to the X-ray marker areas 42, 43, 44, 45, 46. The X-ray marking 21, 26 may be overlaid on the X-ray marker areas 42, 43, 44, 45, 46 or may be arranged beneath or around these. The spectral absorption properties of the X-ray marking 21, 26 enable identification or differentiation of the instruments or needles 41 if a number (e.g., several) are shown in the tomographic image 40.

Gender-neutral language is used throughout this text.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A medical instrument for invasive use, the medical instrument comprising:
an X-ray marker arrangement comprising at least two X-ray marker areas,
wherein the at least two X-ray marker areas are configured to have different absorption properties for X-radiation with different energy spectra, and
wherein the at least two X-ray marker areas are arranged successively on the medical instrument with regard to a predetermined spatial direction.

2. The medical instrument of claim 1, wherein the medical instrument is configured in elongated form, and the predetermined spatial direction corresponds to a longitudinal direction of the medical instrument.

3. The medical instrument of claim 1, wherein each of the at least two X-ray marker areas has a different metal content.

4. The medical instrument of claim 1, wherein the at least two X-ray marker areas are configured as X-ray markers spatially separated from each other.

5. The medical instrument of claim 1, wherein the at least two X-ray marker areas are configured as areas of a one-piece X-ray marker.

6. The medical instrument of claim 5, wherein differences in absorption properties of the at least two X-ray marker areas vary incrementally for X-radiation with different energy spectra from X-ray marker area to X-ray marker area of the at least two X-ray marker areas.

7. The medical instrument of claim 1, further comprising at least one other X-ray marking that is configured to have different absorption properties for X-radiation with different energy spectra and is arranged to spatially encompass the X-ray marker arrangement.

8. A method for determining positioning information for an invasive medical instrument, the method being computer-implemented and comprising:
receiving a tomographic image by a computer unit;
detecting at least one instrument in the tomographic image;
determining a spatial position of the at least one instrument in the tomographic image;
receiving an X-ray image data set comprising the spatial position of the at least one instrument, which is based on at least two X-ray images that were produced with X-radiation with a different energy spectrum in each case;
determining at least one parameter value from the X-ray image data set for the spatial position of the at least one instrument, wherein the at least one parameter value depends on different absorption properties of the at least one instrument or of an X-ray marker area of the at least one instrument for X-radiation with different energy spectra;
determining positioning information assigned to the respective parameter value; and
outputting the positioning information or information derived from the positioning information via an output device.

9. The method of claim 8, wherein determining the positioning information comprises obtaining the positioning information from a database, and
wherein the positioning information is related to the spatial position of a respective X-ray marker area on the at least one instrument.

10. The method of claim 8, wherein determining the at least one parameter value comprises determining at least two parameter values for different points on the spatial position of the at least one instrument,
wherein the at least two parameter values depend on the different absorption properties of a respective X-ray marker area of the at least one instrument for X-radiation with different energy spectra,
wherein determining the positioning information comprises determining the positioning information using the at least two parameter values, and
wherein the positioning information is related to the spatial position of the respective X-ray marker areas of the at least one instrument with regard to each other.

11. The method of claim 8, further comprising determining, in addition to the parameter value or values of the at least one instrument or of the respective X-ray marker areas of the at least one instrument, at least one parameter value for an X-ray marking,
wherein the at least one parameter value depends on different absorption properties of the X-ray marking for X-radiation with different energy spectra, and
wherein identification information assigned to the parameter value is determined, which is related to identification of the at least one instrument.

12. An X-ray device comprising:
a computer unit comprising a processor configured to determine positioning information for an invasive medical instrument, the determination of the positioning information for the invasive medical instrument comprising:
receipt of a tomographic image by a computer unit;
detection of at least one instrument in the tomographic image;
determination of a spatial position of the at least one instrument in the tomographic image;
receipt of an X-ray image data set comprising the spatial position of the at least one instrument, which is based on at least two X-ray images that were produced with X-radiation with a different energy spectrum in each case;
determination of at least one parameter value from the X-ray image data set for the spatial position of the at least one instrument, wherein the at least one parameter value depends on different absorption properties of the at least one instrument or of an X-ray marker area of the at least one instrument for X-radiation with different energy spectra;
determination of positioning information assigned to the respective parameter value; and
output of the positioning information or information derived from the positioning information via an output device.

13. In a non-transitory computer-readable storage medium that stores instructions executable by one or more processors to determine positioning information for an invasive medical instrument, the instructions comprising:
receiving a tomographic image by a computer unit;
detecting at least one instrument in the tomographic image;
determining a spatial position of the at least one instrument in the tomographic image;
receiving an X-ray image data set comprising the spatial position of the at least one instrument, which is based on at least two X-ray images that were produced with X-radiation with a different energy spectrum in each case;
determining at least one parameter value from the X-ray image data set for the spatial position of the at least one instrument, wherein the at least one parameter value depends on different absorption properties of the at least one instrument or of an X-ray marker area of the at least one instrument for X-radiation with different energy spectra;
determining positioning information assigned to the respective parameter value; and
outputting the positioning information or information derived from the positioning information via an output device.

14. The non-transitory computer-readable storage medium of claim 13, wherein determining the positioning information comprises obtaining the positioning information from a database, and
wherein the positioning information is related to the spatial position of a respective X-ray marker area on the at least one instrument.

15. The non-transitory computer-readable storage medium of claim 13, wherein determining the at least one parameter value comprises determining at least two parameter values for different points on the spatial position of the at least one instrument,
wherein the at least two parameter values depend on the different absorption properties of a respective X-ray marker area of the at least one instrument for X-radiation with different energy spectra,
wherein determining the positioning information comprises determining the positioning information using the at least two parameter values, and
wherein the positioning information is related to the spatial position of the respective X-ray marker areas of the at least one instrument with regard to each other.

16. The non-transitory computer-readable storage medium of claim 13, wherein the instructions further comprise determining, in addition to the parameter value or values of the at least one instrument or of the respective X-ray marker areas of the at least one instrument, at least one parameter value for an X-ray marking,
wherein the at least one parameter value depends on different absorption properties of the X-ray marking for X-radiation with different energy spectra, and
wherein identification information assigned to the parameter value is determined, which is related to identification of the at least one instrument.
